(19) United States
(12) Patent Application Publication
(10) Pub. No.: US 2022/0409440 A1
(43) Pub. Date: Dec. 29, 2022

(54) PROCESS FOR INTEGRATION OF A SIGNAL CONNECTION, IN AN INCONTINENCE SENSING PRODUCT, USING A UNI-FASTENER INTO A HIGH-SPEED MANUFACTURING LINE

(71) Applicant: Daniel Ross Collette, Albuquerque, NM (US)

(72) Inventor: Daniel Ross Collette, Albuquerque, NM (US)

(21) Appl. No.: 17/360,936

(22) Filed: Jun. 28, 2021

(51) Int. Cl. A61F 13/15; A61F 13/42

(52) U.S. Cl. CPC A61F 13/15756; A61F 13/42

(57) ABSTRACT

This patent describes the design of a novel metal snap, either ball or socket, and the process by which it is integrated and connected to conductive elements in incontinence products, manufactured in high speed production lines, which makes possible the monitoring and detection of incontinent events in the product.

### CROSS REFERENCE TO RELATED APPLICATIONS

This application claims the priority of Provisional Patent Applications No. 63/045,077 filed Jun. 27, 2020, the entire contents of which are incorporated by reference herein. U.S. Pat. No. 8,421,636 B2 Patient Monitoring System, referenced because it contains information and materials related to sensor strips placed in incontinence articles.

### STATEMENT REGARDING FEDERALLY SPONSORED RESEARCH OR DEVELOPMENT

Not Applicable

### BACKGROUND OF THE INVENTION

### 1. Field of the Invention

The present invention relates to the design, function and integration of a novel metal socket and the associated process by which it is integrated and connected to conductive elements in incontinence products, which are manufactured in high-speed production lines. This advance in 20-year-old technology will make possible the mass production of hundreds of different incontinence products used in hospitals, recovery centers, assisted living facilities, nursing homes, in-home care and millions of people who are caring for handicapped children and aging parents in their own homes.

### 2. Description of the Related Art

The earliest patent issued for the introduction of sensor strips in incontinence products was patented in U.S. Pat. No. 8,421,636 issued Apr. 16, 2013. Since that point in time, there have been numerous variations of attempted connections to these sensors and other sensors of competing materials and styles. Connection methods have included snaps, clips and pin arrays as well as a myriad of others attempts. Sockets or posts (one type of snap assembly) have emerged as the preferred method due to the ease of connection (everyone knows how to connect a snap) and the manual setting of these snaps with a snap setting machine ensures good placement and contact with the sensor strips.

Because the sensor strips in high-speed manufacturing have to have significant tensile strength to preserve the continuity of the aluminum, it is backed with a mylar transport film. The mylar film while providing excellent carrier properties, it poses many difficulties in making good contact to the world outside of the incontinence product.

Some alternate methods of attachments to incontinence products are needles, pins, clips of various configurations and compression. Pins or other puncture or clip type of contact to the sensor strip have proven in years of study to actually prevent effective contact, because the process of piercing the polymer, carries or splinters small particles of the carrier and imposes a non-conductive poly material between the sensor conductive element and the pins or other puncture or clip methods. There are significant complications with each of these connectivity methods, including, health risks from puncture wounds, inconsistent connections, shorts or opens.

Snaps on the other hand are easy to connect to and don't have any of these connectivity issues. The shape of prongs and manner of clamping in press fitting those prongs into posts or sockets has shown nearly 100% connectivity for transmission of signals.

Snap Fasteners

The background and uses of snaps are well understood and universal around the world. Set-in Snap fasteners (hereafter referred to as Snap Fasteners) have been in existence for a long time. The first patent for a snap fastener was issued to Heribert Bauer in 1885, a fastener for trousers.

Snap Fasteners have multiple components. The male component, referred to as a ball or stud (hereafter referred to as the Stud), has a corresponding piece called an Open Prong (hereafter referred to as a Prong), which when placed on the opposite side of the material and compressed, attaches the Stud to the object. The female component of a snap, referred to as a Socket, also has a corresponding piece, called a Snap Cap (hereafter referred to as a Snap) which is compressed through the material where it is to be placed, binding the snap to the material. The female component could also have a Prong as it's complementary component.

Set-in Snap fasteners are used in a variety of applications . . . to fasten clothing, purses, baby clothes, camping equipment, incontinent product sensing. The list of uses is endless. Pronged rivets are also well known and used significantly to attach decorative jewels, beads and other items to clothing. Both a snap fastener and pronged rivets have been around for over 100 years. Although similar at first glance, the utility of a pronged rivet is completely different than the utility of a uni-fastener as described in this patent. Rivet prongs and snaps are used as fasteners of decorative bobbles or on clothing, belts and other accessories as closures. In a belt, for example, rivets are used to bind the tail of the belt after passing through the buckle, back to the belt itself to ensure that it doesn't pull back through the buckle. Prongs on paper fasteners, are pushed through paper and then bent outward to bind the sheets of paper together. Prongs are found on rhinestones and decorative fasteners on clothing. Prongs are found on T-nuts and other types of furniture. From all these examples, we see that it is not the prong, but rather the form and function of what has the prong that is critical and the determining factor of form fit and function.

Disposable diapers were introduced by Johnson and Johnson in 1948. Although not widely known, standard snap fasteners have been used incontinent products, to provide a simple but effective attachment to sensor strips in the incontinent product. This allows the simple attachment of a miniature wireless sensor to the incontinent article.

For almost 20 years (2003), disposable sensing incontinence products have had snap fasteners used to functionally connect a sensing or detection device to the incontinent product. Because of the speed at which incontinence products are manufactured, the placement of a conventional snap for connectivity has not ever been done in a highspeed manufacturing line. The snap setters are heavy and bulky and cannot move at a meter/sec and get back in time to place the next snap.

The placement of snap fasteners in current incontinent sensing products, has been accomplished by using a manual snap setter machine to attach the snap fasteners in a finished sensory incontinence product, one snap at a time. This has provided a very reliable electrical connection to the sensory product, but at a significant cost in labor.

The standard two-part snap fasteners, currently used as a connection means in incontinent products, also has some significant drawbacks, resulting from the interior prong ring (image) in contact with skin surfaces, or moisture, causing false signals or skin irritation. This issue has necessitated the application of a paper tape over the interior prong ring in order to avoid touch skin and these significant drawbacks. This also significantly increases labor and cost of the product.

The single most important impediment to the introduction of sensory incontinent products into the market place has been connectivity and manufacturing constraints associated with that connectivity. The drawback for snaps is that they cannot be integrated into high-speed manufacturing, because the line is moving too fast to do the alignment and compression required to set the snaps, and move back to the starting position in time to set the next snap.

The uniqueness of this pronged socket, concept and design, originating from significant amounts of field trials and years of gathering data was the process of first maintaining a connection that everyone was familiar with the usage and making it so the pronged socket would run in a process that would embed the prongs of the socket in the article in such a way that it could not touch wearer skin, and allow the pronged socket to be placed in articles where manufacturing changes have made it impossible to attached to what had been a previously exposed sensor strip at the waist of a diaper. See FIG. 1.

As diapers have moved towards a pant like diaper, the processing required to make this product, no longer permitted the placement of a normal snap because the sensor strips are cut at the edge of the fluff and absorbent layers in the manufacturing process, thus inhibiting a snap placed in post processing to be able to contact the metallic side of the sensing strip so that proper electrical contact was made.

Therefore, this patent claims not only the unique and non-obvious design of the pronged socket, but also the unique and non-obvious process required to introduce, attach the socket to an incontinence product of every kind, in the high-speed manufacturing lines used today by every major manufacturer.

As shown earlier, prongs are found on many different and unique articles. It is not the prong that is unique, but the items and even more importantly, the function of the items the prongs attach to that is unique. The prongs on a bobble are like the prongs on a rivet and are like the prongs on a T-nut but yet it is the function that is unique and critical and patentable. The placement of a bobble on a piece of clothing, a rivet binding a buckle on a belt, the rivets preventing a T-nut from turning when a bolt is inserted and tightened, or a socket from being connected to a sensor strip in an incontinence product.

Unlike all other snap or prong devices, this pronged socket, has multiple purposes beyond just fastening. The prongs are critical in making good contact to the sensor strips. Even more important is the fact that the method of placing the socket in contact with the sensor strips within the restrictions of a fast-moving manufacturing line and then embedding it, is novel and unique. No existing snap or prong has this purpose and so this patent stands apart in function and therefore is unique from a two-piece snap and prong socket and cannot either function or be incorporated into the product on a fast-moving line without the novel configuration shown in this patent.

### BRIEF SUMMARY OF THE INVENTION

The present invention relates to the design, function and integration of a novel metal pronged socket and the associated process by which it is integrated and connected to conductive elements in incontinence products, which are manufactured in high-speed production lines.

In the normal use for snaps, the post or socket is on one side of the material or garment and the prongs, caps or posts on the opposite side. The complementary pieces are aligned and pressed together to make a finished snap. This provides a nice finished product, and the durability of thousands of snaps and unsnaps with failure.

The manufacture of incontinence products, is in a high-speed process where the material is travelling at one meter per second. At this speed, it was determined by the engineers at the largest US manufacturer for medical and health supplies, including incontinence products, that it was impossible on their incontinence lines, to move the mass of the press which both aligns and compresses the two components of a standard snap and then move back to the starting position in time for the next placement. They abandoned the project.

Therefore, it was necessary to develop a novel process and a subsequent novel socket design that could be used together in the proposed process.

The socket had to be one piece!

The process of installing the snap in a high-speed manufacturing line is as follows: (this varies by manufacturer and product, but is general and valid, that it can be incorporated into high-speed manufacturing lines.
1) The first layer (impermeable) is laid down.
2) This is followed by an adhesive spray to adhere subsequent fluff and absorbent material.
3) The adhesive spray is followed by the introduction of the sensor strips, consisting of a thin aluminum film on a mylar backing. The sensor strips must be contacted by the exterior single piece snap. The sensor strips are introduced usually from the right or left side of the flow, perpendicular to the moving material, passing a set of angled rollers which rotate the tape 90 degrees and invert in preparation to being placed in the moving product. This is a well-established process.
4) The single piece snap with prongs is delivered on a carrier medium, prongs pointing upward, mounted and spaced correctly for the sensor strip placement, against the outside of the impermeable layer. The carrier medium is controlled to flow at the same speed as the manufacturing line and is variable. A brush roller on the opposite and interior side where the aluminum sensor film is in place, puts downward pressure on the impermeable layer and sensor strips forcing the prongs to push through the impermeable layer and the sensor strips. The brush roller presses the layers down, but at the same time allows the prongs of the One-Piece Snap to come up into the bristles of the brush roller. This can be accomplished by any means that forces the material down into the prongs of the socket such as a burst of air.
   5) Immediately after the bush roller, the prongs are flattened by rollers or instantaneous piston or other compression techniques.
   6) The carrier pulls away from the line and picks up another socket.

A completed incontinence product with sensing strips in shown in FIG. 4.

There is another advantage to this process, and it is important and a key element in the uniqueness of this process, which is that since there is only a single part to this snap, once the initial layers are put down in the process, and the prongs are flattened, they become immediately embedded inside the article as they are then covered with the fluff and absorbent materials of the further processing and the issue with skin contact and errant notifications are no longer possible.

It also makes it possible to put the snaps below the top of the finished article, such as wearable disposable pant diapers, where the stretch waist exists and where the sensor strips are cut in the process at the edge of the pulp, making it impossible to use a two piece snap setting operation, since there is no exposed sensor tape to attach to in these newer wearable products, including adult wearable incontinent products, post manufacture.

### BRIEF DESCRIPTION OF THE DRAWINGS

FIG. 1 is a drawing of a pronged socket.

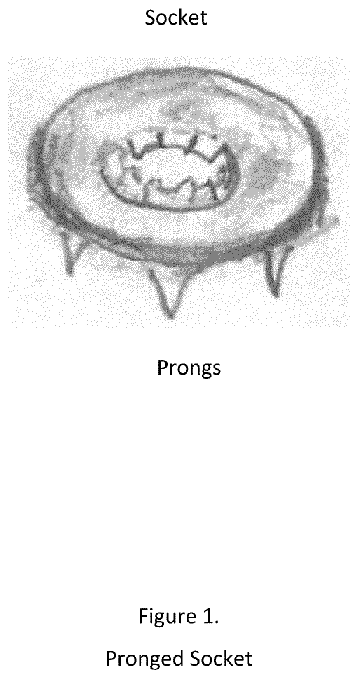

FIG. 2 is a drawing of pronged sockets on a delivery tape. There are two rows of sockets because there are two sensor strips in the incontinence products and so there must be two sockets presented at the same time, one socket for connection to each sensor line.

FIG. 3 is the process flow for integration of the pronged sockets to the incontinent product.

FIG. 4 shows the attachment of two sockets to the sensor strips and shows a completed incontinent product with sensor strips and sensor module attached to sockets

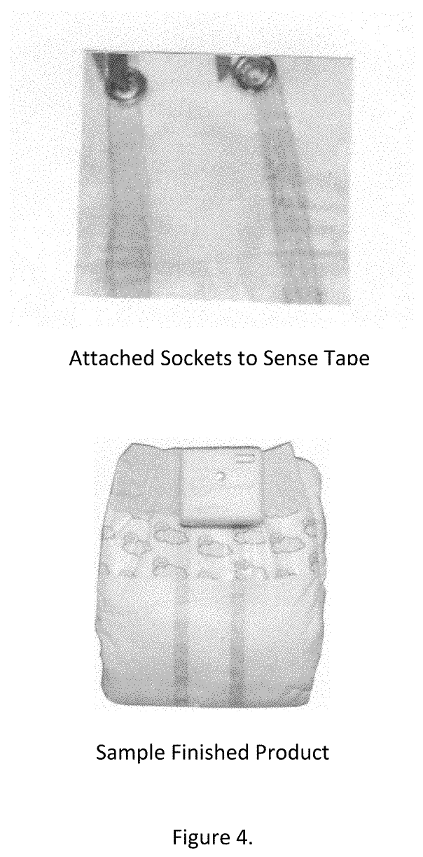

FIG. 5. Shows renditions of a one-piece pronged socket and a one-pieces pronged stud.

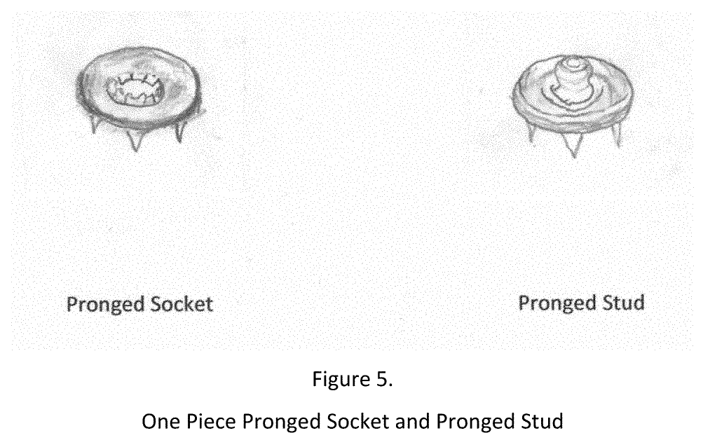

What is claimed is:

1. A method for integrating an external connection interface to the sense lines of an incontinence article on a high-speed manufacturing line for incontinence articles.

2. A one-piece snap socket, comprising a metal snap socket with two or more prongs antipodean to the socket face.

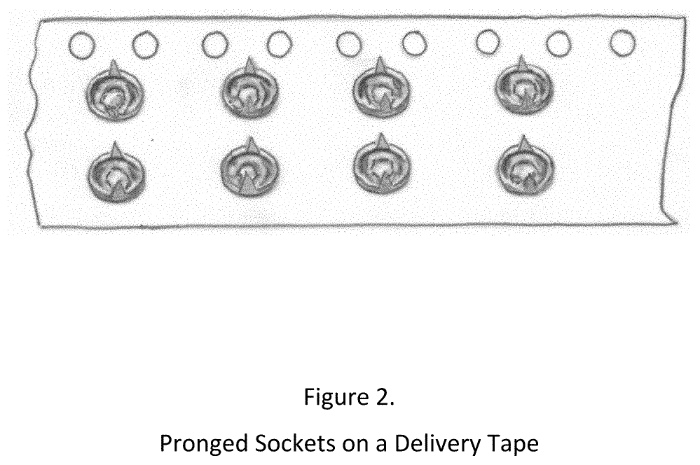

3. A carrier tape manufactured and designed for delivery of metal snap sockets with prongs as shown in FIG. 2, comprising a carrier tape with snap posts which will pick up the one-piece snap sockets according to claim 2.

4. A high-speed manufacturing line for incontinence products comprising these additional processing steps and accompanying equipment; a. rollers and guides to introduce an aluminum backed mylar sensing tape; b. rollers and guides to introduce the one piece snap socket on a delivery tape; c. a brush roller or air jet for prong insertion; d. An instantaneous press or roller for compressing prongs; e. rollers and guides to exit the socket delivery tape.

5. A method for integrating an external connection interface according to claim 1, further comprising the once piece snap socket according to claim 2, further comprising a carrier tape according to claim 3, further comprising a high-speed manufacturing line for incontinence products according to claim 4.

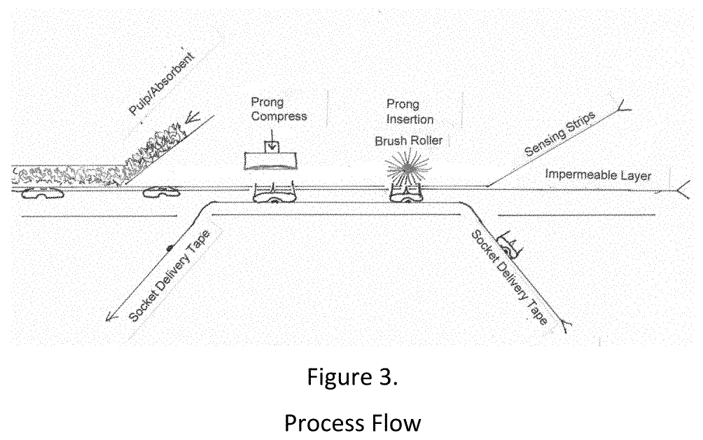

6. A method for integrating an external connection interface according to claim 5 where the following sequential steps occur. First, the carrier tape in claim 3, which is synchronized with the line speed, is introduced below the impermeable layer as shown in FIG. 3. The prongs on the socket are up facing the exterior surface of the impermeable layer. When the sockets pass beneath the prong insertion point as shown in FIG. 3, the brush roller or air jets, press the impermeable layer with the accompanying sensing strips downward, causing the prongs to pierce both layers such that the prongs are fully through the layers and against the socket. The line continues to move forward and at the point when the socket is beneath the prong compress, as shown in FIG. 3, the press instantaneously closes, compresses the prongs fast to the sensor tape and the socket body, providing good contact between the sensor tape and the exterior socket, via the prongs. As the line continues to move, the carrier tape is separated from the socket while the product continues to move into the pulp deposition, which buries the prongs and prevents skin and sweat contact from sending erroneous signals.

7. A method for integrating an external connection interface according to claim 6, where the pronged sockets in claim 2 are replace by the pronged socket studs as indicated in claim 8.

8. A one-piece snap post, comprising a metal snap post with two or more prongs antipodean to the post face.
